(19) United States
(12) Patent Application Publication
(10) Pub. No.: US 2020/0037917 A1
(43) Pub. Date: Feb. 6, 2020

(54) DEPLOYABLE LOCAL MAGNETIC RESONANCE IMAGING COIL AND METHODS FOR USE THEREOF

(71) Applicant: The Brigham and Women's Hospital, Inc., Boston, MA (US)

(72) Inventors: Ehud J. Schmidt, Newton, MA (US); Thomas Johnson, New Ipswich, NH (US); James Wright, Boston, MA (US); Erin McKenna, Boston, MA (US); Joseph Ting, Acton, MA (US)

(21) Appl. No.: 16/340,283

(22) PCT Filed: Oct. 11, 2017
(86) PCT No.: PCT/US2017/056215

§ 371 Date: Apr. 8, 2019

Related U.S. Application Data
(60) Provisional application No. 62/407,327, filed on Oct. 12, 2016.

(51) Int. Cl. A61B 5/055; G01R 33/34; G01R 33/28; G01R 33/36; A61B 5/00

(52) U.S. Cl. CPC A61B 5/055; G01R 33/34084; G01R 33/285; A61B 5/6853; G01R 33/34007; A61B 5/6851; G01R 33/3621

(57) ABSTRACT

A catheter device for deploying a local magnetic resonance imaging coil is provided. The catheter device comprises an inner shaft, an outer sheath, and a local magnetic resonance imaging coil. The outer sheath includes a plurality of slits extending in an axial direction proximate a distal end of the outer sheath. The slits separate a portion of the outer sheath into a plurality of struts. The local magnetic resonance imaging coil is disposed between the inner shaft and the outer sheath and is coupled to the plurality of struts. Moving the outer sheath relative to the inner shaft expands the catheter device from a contracted position to an expanded position.

### CROSS-REFERENCE TO RELATED APPLICATIONS

This application claims the benefit of, and incorporates herein by reference in its entirety, U.S. Provisional Application 62/407,327 filed Oct. 12, 2016.

### STATEMENT REGARDING FEDERALLY SPONSORED RESEARCH

This invention was made with government support under U54HL119145 awarded by the National Institutes of Health. The government has certain rights in the invention.

### BACKGROUND

The present disclosure relates generally to an apparatus for use as a local magnetic resonance imaging (“MRI”) coil in an MRI system. MRI coils are used as transmitters and receivers for MRI signals, for monitoring the delivery of thermal ablation (inductive heating of tissues, such as used in cardiac electro-physiology), and in conjunction with the transmission and reception of electric-magnetic waves propagating in the body at a variety of frequencies and conditions (EEG, ECG, nerve conduction, muscle fibrillation and defibrillation, etc.). Typically, MRI coils are sensitive to distances which are proportional to their dimensions, with larger coils being effective for larger distances. Effective use of MRI coils is difficult within the human body.

Introduction of MRI coils into the body, whether through body orifices, through the vascular tree, or through man-made punctures created during surgery/intervention, presents a constraint of requiring that such coils must go into the body through small channels, frequently an order of magnitude or more smaller than their desired deployment size. For example, in the Intra-cardiac MRI (“ICMRI”) catheter case, it is desired to image the walls of the heart at high sensitivity using MRI imaging methods, without touching or damaging the walls. This calls for using deployed MRI coil sizes with a diameter of between about 20 mm and 30 mm. However, the MRI coils are introduced into the heart on a catheter that can be navigated through the vascular tree, which requires that the MRI coils have an initial configuration with a diameter of between about 3 mm and 4 mm during the navigation stages (insertion and withdrawal), so as to prevent damaging any portion of the vascular anatomy.

Providing an MRI coil capable of expanding from a small size (during navigation) to a much larger size (when they are deployed) calls for having effective means to: (1) selectively contract the MRI coil into a smaller compacted configuration and (2) selectively expand the MRI coil to a larger expanded configuration. Additionally, operations (1) and (2) need to be performed without damaging the mechanical properties (e.g., integrity, flexibility, elastic moduli, shape of device) and electrical properties (e.g., radio-frequency sensitivity, conductivity, and permittivity) of the MRI coils.

It would therefore be desirable to provide an apparatus for use as a local MRI coil in an MRI system that is capable of being selectively expandable, without damaging the mechanical and electrical properties of the local coil. Furthermore, it would be desirable to provide a method of making such an apparatus that is less expensive to assemble and requires less time to manufacture and maintain.

### SUMMARY

The present disclosure provides systems and methods that overcome the aforementioned difficulties by providing a catheter device for deploying a local magnetic resonance imaging coil that is selectively expandable that is both less expensive to assemble and requires less time to manufacture and maintain than traditional expandable catheter-based systems.

In accordance with one aspect of the disclosure, a catheter device for deploying a local magnetic resonance imaging coil is provided that comprises an inner shaft, an outer sheath, and a local magnetic resonance imaging coil. The outer sheath envelops the inner shaft and includes a plurality of slits extending in an axial direction proximate a distal end of the outer sheath. The slits separate a portion of the outer sheath into a plurality of struts. The local magnetic resonance imaging coil is disposed between the inner shaft and the outer sheath and is coupled to the plurality of struts. Moving the outer sheath relative to the inner shaft expands the catheter device from a contracted position to an expanded position. When the catheter device is in the expanded position, the struts are bent radially outwards away from the inner shaft expanding the local magnetic resonance imaging coil from a contracted position to an expanded position.

The catheter device can further comprise a handle including a sliding member rigidly fixed to the outer sheath. The sliding member can be configured to move the outer sheath relative to the inner shaft to expand the catheter device from the contracted position to the expanded position. The inner shaft and the outer sheath can be rigidly fixed relative to each other at a distal end of the catheter device. The catheter device can further comprise a plurality of microfilaments coupled to the outer sheath near a proximal end of the plurality of struts and near a distal end of the plurality of struts. The microfilaments can each be coupled to the local magnetic resonance imaging coil and can be configured to aid in collapsing the local magnetic resonance imaging coil when the local magnetic resonance imaging coil is moved from the expanded position to the contracted position. The catheter device can further comprise a plurality of motion tracking coils coupled to the plurality of struts. The local magnetic resonance imaging coil can further include tuning and matching capacitors. The local magnetic resonance imaging coil can be configured to be in communication with a radio frequency system of a magnetic resonance imaging system. The local magnetic resonance imaging coil can be coupled to a substrate including a plurality of cutouts. The plurality of cutouts can locally reduce a width of the substrate, providing a plurality of natural bend points in the substrate configured to aid in collapsing the local magnetic resonance imaging coil when the local magnetic resonance imaging coil is moved from the expanded position to the contracted position. The inner shaft can further include a lumen sized to receive a medical device. The medical device can comprise at least one of a radio frequency ablation (RFA) catheter, a laser ablation catheter, a cryoablation catheter, a guide wire, a stenting catheter, and a balloon angioplasty catheter. The local magnetic resonance imaging coil can further include a small (“micro”) low-noise preamplifier wafer, as well as anti-parallel diode wafers that protect the preamplifier from damage due to strong radio-frequency signals, configured to amplify the signal without increasing its noise and thereby reduce electrical noise in a radio frequency system, especially since the radio frequency signal is conducted up the catheter shaft through thin electrical transmission lines, which can increase the radio frequency noise, so amplification of the signal at its source enables lower noise reception, and substantially improves (5-6 times) the signal to noise ratio of the received signal from the imaging radio frequency coil.

In accordance with another aspect of the disclosure, a catheter device for deploying a local magnetic resonance imaging coil is disclosed that comprises an inner shaft, an outer sheath, and a local magnetic resonance imaging coil. The outer sheath includes a plurality of slits extending in an axial direction proximate a distal end of the outer sheath. The slits separate a portion of the outer sheath into a plurality of struts. The local magnetic resonance imaging coil is disposed between the inner shaft and the outer sheath and is coupled to the plurality of struts. Moving the outer sheath relative to the inner shaft expands the catheter device from a contracted position to an expanded position.

The catheter device can further comprise a handle including a sliding member rigidly fixed to the outer sheath. The sliding member can be configured to move the outer sheath relative to the inner shaft to expand the catheter device from the contracted position to the expanded position. The inner shaft and the outer sheath can be rigidly fixed relative to each other at a distal end of the catheter device. The catheter device can further comprise a plurality of microfilaments coupled to the outer sheath near a proximal end of the plurality of struts and near a distal end of the plurality of struts. Each of the plurality of microfilaments can be coupled to the local magnetic resonance imaging coil and can be configured to aid in collapsing the local magnetic resonance imaging coil when the local magnetic resonance imaging coil is moved from the expanded position to the contracted position. The local magnetic resonance imaging coil can be coupled to a substrate. The substrate can include a plurality of cutouts, which locally reduce a width of the substrate, providing a plurality of natural bend points in the substrate. The plurality of natural bend points can be configured to aid in collapsing the local magnetic resonance imaging coil when the local magnetic resonance imaging coil is moved from the expanded position to the contracted position. The inner shaft can further include a lumen sized to receive at least one of a radio frequency ablation catheter, a laser ablation catheter, a cryoablation catheter, a stenting catheter, and a balloon angioplasty catheter.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1A is an elevation view of an exemplary catheter device including a local magnetic resonance imaging (“MRI”) coil, shown with the catheter device in a fully contracted position.

FIG. 1B is an elevation view of the exemplary catheter device of FIG. 1A, shown with the catheter device in an expanded position.

FIG. 2 is a cross-sectional view of a handle housing of the catheter device of FIGS. 1A and 1B.

FIG. 3 is an elevation view of an exemplary expandable local MRI coil that forms part of the catheter device of FIGS. 1A and 1B.

FIG. 4 is an elevation view of another exemplary expandable local MRI coil that can be used with the catheter device of FIGS. 1A and 1B, which includes a preamplifier wafer.

FIG. 5 is a schematic diagram of a DC powering circuit of the preamplifier wafer of the expandable local MRI coil of FIG. 4.

FIG. 6 is a schematic diagram of a circuit of a preamplifier wafer and its DC powering circuit for use with the local MRI coil of FIG. 4.

FIG. 7A is a detailed view of the catheter device of FIGS. 1A and 1B, shown with the catheter device in the contracted position, including microfilaments shown in dotted lines.

FIG. 7B is a detailed view of the catheter device of FIG. 1B, shown with the catheter device in the expanded position, including the microfilaments shown in solid and dotted lines, and shown without the local MRI coil.

FIG. 8A is a detailed view of the catheter device of FIGS. 1A and 1B, shown with the catheter device in the expanded position.

FIG. 8B is a detailed view of the catheter device of FIGS. 1A and 1B, shown with the catheter device in a partially contracted position.

FIG. 8C is a detailed view of the catheter device of FIGS. 1A and 1B, shown with the catheter device in a further contracted position.

FIG. 9 is a block diagram of an example of a magnetic resonance imaging (“MRI”) system configured for use with the catheter device illustrated in the preceding figures.

### DETAILED DESCRIPTION

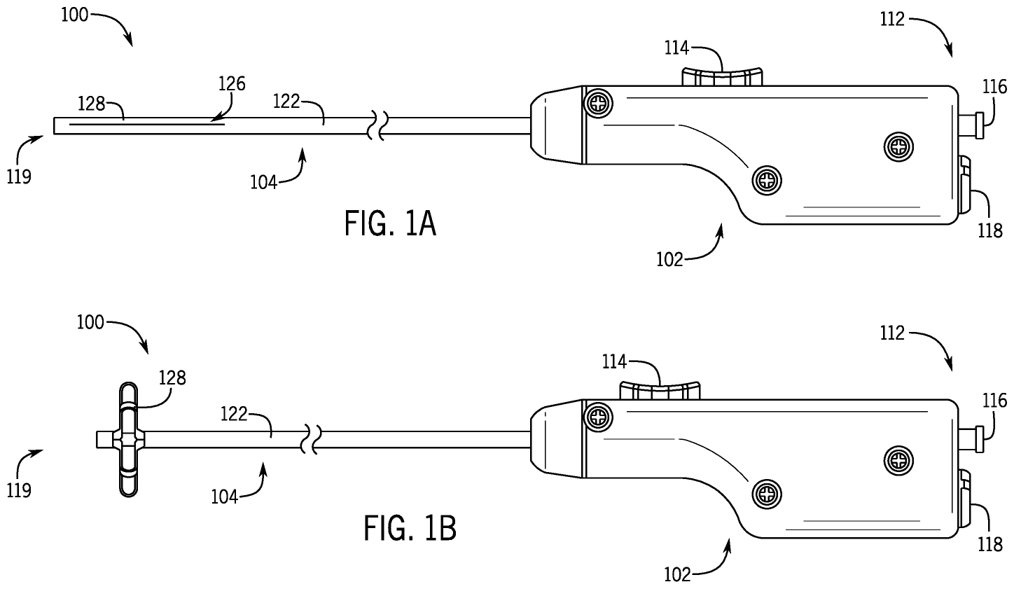

By way of overview and introduction, an expandable catheter device 100 that can provide imaging and/or motion tracking coils for magnetic resonance imaging (“MRI”) is generally illustrated in FIGS. 1-8C. As will be described, one advantageous clinical use of the expandable catheter device 100 is intra-cardiac medical procedures, such as thermal ablation procedures and the like. The expandable catheter device 100 can be used to monitor a therapy, such as a radio frequency ablation (“RFA”) therapy for cardiac atrial fibrillation or ventricular tachycardia, using MRI to acquire images during the therapy so that the therapy can be monitored and adjusted as needed. Furthermore, the catheter device 100 can be designed to effectuate such therapies, to thereby perform a therapeutic process with the catheter device 100, while also monitoring or tracking the therapeutic process and/or providing diagnostic information used to then effectuate the therapeutic process. For example, it is noted that the portion of the radio frequency (“RF”) range employed by RFA is significantly lower than the portion of the radio frequency (“rf”) range employed for MRI. For example, RFA probes commonly operate at frequencies around 500 kilohertz, whereas MRI systems commonly operate at frequencies higher than 20 megahertz. The expandable catheter device 100 can also be used for other vascular and non-vascular treatment applications when MRI imaging can be of benefit, such as the placement of stents and angioplasty balloons. Notably, as used herein, “rf” in lowercase is used to designate radio-frequency energy at the MRI (Larmor) frequency, as opposed to “RF”, which designates radio-frequency energy at the thermal ablation frequency (“RF”, “RFA”, etc.).

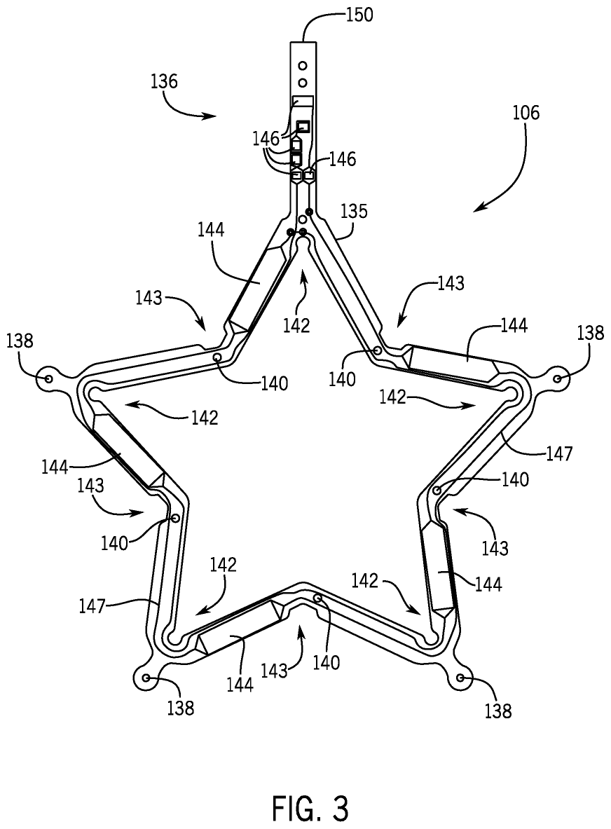

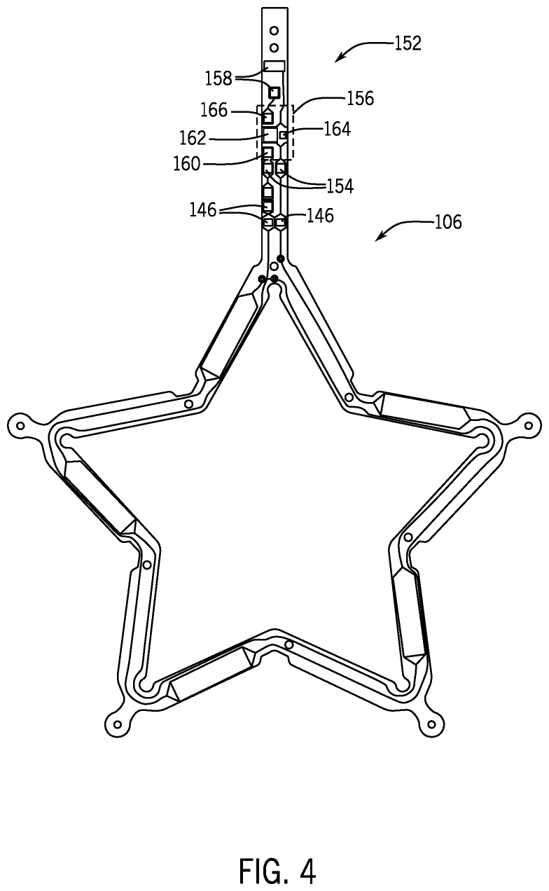

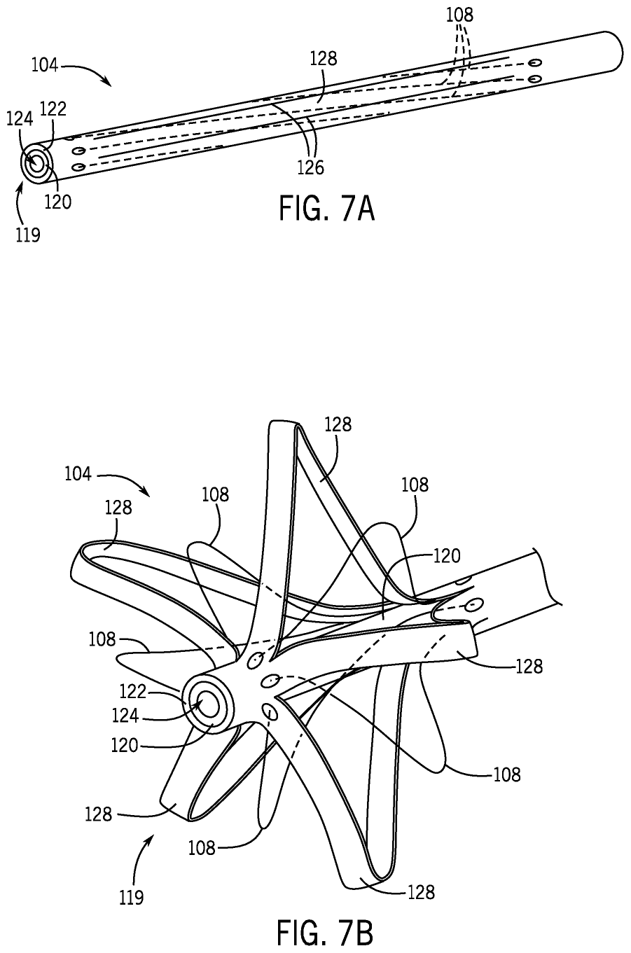

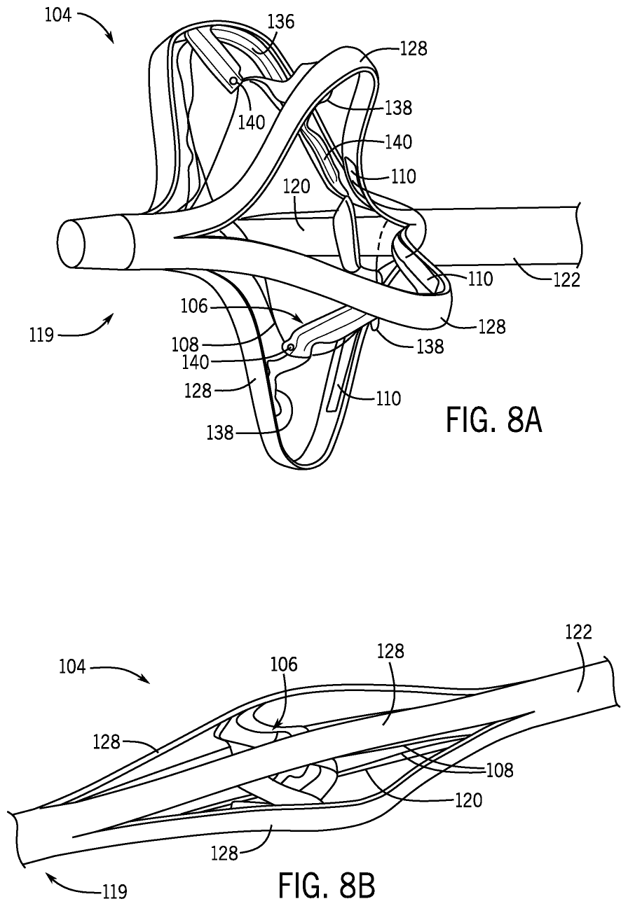

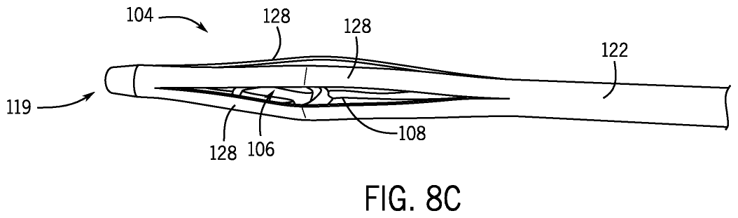

Referring now to FIGS. 1A and 1B, one non-limiting example of the expandable catheter device 100 is illustrated. The expandable catheter device 100 includes a handle 102, a catheter 104, a local magnetic resonance imaging (“MRI”) coil 106 (shown in FIGS. 3 and 4), microfilaments 108 (shown in FIGS. 7A and 7B), and local motion tracking coils 110 (shown in FIG. 8A). The optional local motion tracking coils 110 can be used to perform motion-correction that can be highly advantageous when imaging physiologically moving tissues. As will be described, the local motion tracking coils 110 can employ embedded capacitors for tuning and matching, which provides great flexibility and precision. The handle 102 is disposed on a proximal end 112 of the expandable catheter device 100 and includes a sliding member 114, an inner shaft cap 116, and a signal connection port 118.

The catheter 104 of the expandable catheter device 100 includes an inner shaft 120 (shown in FIGS. 7A and 7B) and an outer sheath 122, which is rigidly fixed to the inner shaft 120 at a distal end 119 of the catheter 104. The inner shaft 120 additionally includes a lumen 124 (also shown in FIGS. 7A and 7B), which may be sized to receive a medical device (not shown), such as an RFA catheter; other electrophysiology ablation catheters, including laser ablation and cryoablation catheters; a stenting catheter; a guide wire, or a balloon angioplasty catheter. The lumen 124 can also be used to extrude liquid at controlled rates to cool the RFA catheter during heating, or to displace tissues, such as cardiac wall tissue, for measurements of 20 elastic constants of the tissues. In the illustrated, non-limiting example, the outer sheath 122 includes five slits 126 disposed near the distal end 119 of the catheter 104 that extend axially along the outer sheath 122. Of course, other numbers of slits 126 may be used. As also illustrated in this non-limiting example, the slits 126 are disposed evenly around the circumference of the outer sheath 122, effectively separating the outer sheath 122 into five generally identical struts 128. However, other arrangements, including non-evenly distributed slits 126 may be used.

Various alternate construction methods for the device may also be used. For example, with respect to the distal section of the outer sheath, a molded tip section could be used. A molded distal tip can be molded in a flat shape with features, such as the slits and holes for the monofilament and the strut profile molded or formed therein, to selectively influence the folding characteristics. The distal tip can also be molded in a tubular form allowing changes to the surface profile that may not be attainable in an extruded tube, such as internal protrusions for securing the flexible circuits. The molded distal tip can be bonded to the proximal section (i.e., the distal end) of the outer sheath to complete the assembly.

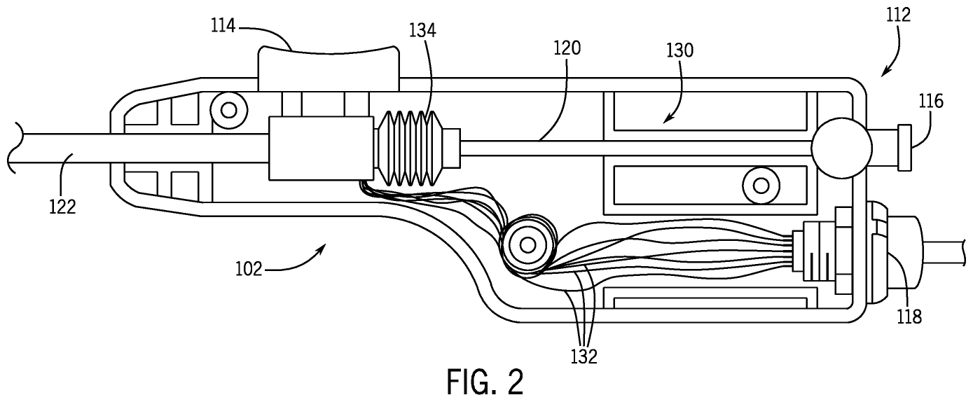

Referring now to FIG. 2, a non-limiting example of an interior portion 130 of the handle 102 is illustrated. The outer sheath 122 extends into the interior portion 130 of the handle 102 and is fixed, for example via a rigid connection, at a proximal end of the outer sheath 122 to the sliding member 114. The sliding member 114 can be transitioned between a proximal position (shown in FIG. 1A) and a distal position (shown in FIG. 1B). Through manipulation of the handle 102 (i.e., by moving the sliding member 114 between the proximal and distal positions), the expandable catheter device 100 is selectively moveable between a fully contracted position (also shown in FIG. 1A) and an expanded position (also shown in FIG. 1B), as will be described in detail below.

The inner shaft 120 extends through the outer sheath 122, through the interior portion 130 of the handle 102, and into the inner shaft cap 116, which is disposed in a proximal end of the handle 102. Several conductive wires 132 extend from the signal connection port 118, which is also disposed in the proximal end of the handle 102. The wires 132 extend through the handle 102, through the inner shaft 120, and into the lumen 124 of the inner shaft 120. The signal connection port 118, in conjunction with the wires 132, provides an electrical connection between the expandable catheter device 100 and the rf system of an MRI system. For example, this connection provides a communication pathway for image data acquired with the local MRI coil 106 and motion tracking data acquired with the tracking coils 110.

The handle 102 may include a bellows 134 attached to and configured to allow relative movement between both the sliding member 114 and the inner shaft 120, while preventing leakage of bodily fluids when the expandable catheter device 100 is in use. Although the exemplary handle 102 includes the bellows 134, other examples can include alternative means of preventing this leakage, such as, for example, O-ring seals, a tapered gasket seal, or viscous sealing materials such as grease.

Referring now to FIGS. 3-6, one non-limiting example of the local MRI coil 106 is illustrated. The local MRI coil 106 is a printed circuit coupled to a substrate 135, which is in the general shape of a five-point star. This five-point star is advantageous for use with the above-described example of the five slits 126. That is, the shape or geometry MRI coil 106 and, as will be described, the substrate 135 coupled with the coil, may be matched to the number of slits 126 and the number of struts 128. Thus, number of slits 126/struts 128 maybe selected based on the desired coil geometry or vice versa. For example, a design with three slits 126/struts 128 could be advantageously coupled with a coil 106 (and substrate 135) in a triangular shape. A design with four slits 126/struts 128 could be advantageously coupled with a coil 106 (and substrate 135) in rectangular shape. A design with six slits 126/struts 128 could be advantageously coupled with a coil 106 (and substrate 135) in a six-pointed star shape. As this correlation of the slits 126/struts 128 with advantageously selected a coil 106 (and substrate 135) geometries can continue toward larger numbers, the shape of the coil approaches or becomes circular. This has the disadvantage of having increasing material constraints to be managed and, instead, one may choose a coil design such as described in U.S. Pat. No. 8,983,574 that manages materials constraints differently.

The substrate 135 includes a tail section 136, strut attachment portions 138, and microfilament attachment portions 140. The tail section 136 is disposed at one of the outer vertices with the strut attachment portions 138, being disposed at each of the other outer “points”. The microfilament attachment portions 140 are each then disposed proximate a corresponding one of the inner vertices.

The substrate 135 further includes inner cutouts 142 disposed at the inner side of each outer vertex or “point” of the star shape and outer cutouts 143 disposed at the outer side of each inner vertex or point of the star shape. The cutouts 142, 143 narrow the local width of the substrate 135, effectively providing the substrate 135 with several natural bending points. These bending points further facilitate the local MRI coil 106 collapsing into a tightly compressed configuration when the expandable catheter device 100 is moved into the contracted position, as will be described below in detail.

Again, it should be appreciated that, although the substrate 135 is illustrated in this non-limiting example in the general shape of a five-point star, the substrate 135 could take other forms, such as, for example, a star with more or less than five vertices, or other shapes generally and need not be matched to the number of slits 126 and struts 128.

Additionally, the local MRI coil 106 may include embedded capacitors 144 and tuning and matching capacitors 146. The embedded capacitors 144 may be disposed around the periphery of the local MRI coil 106 and the tuning and matching capacitors 146 may be disposed within the tail section 136 of the substrate 135. In rf coils that operate at high frequencies, where electromagnetic wave conduction is restricted to a skin depth of only a few microns, the embedded capacitors which are employed along the transmission lines serve to increase the conductive surface area of the rf coils, thus lowering the rf coil's ohmic impedance (or loss factor) and thereby improving the signal to noise ratio of the coils. However, other locations may also be used or the capacitors, in some designs, may be foregone from the proximity of the coil. In the illustrated configuration, the embedded capacitors 144 may each include two conductive pads separated by a dielectric film (not shown). In the illustrated example configuration, the embedded capacitors 144 and the tuning and matching capacitors 146 of the local MRI coil 106 are connected by printed wiring 147. In the illustrated non-limiting example, the conductive pads and the printed wiring 147 are made of a flexible copper material. However, the conductive pads can additionally or alternatively be made of gold, aluminum, copper-nickel alloys, beryllium-copper alloys, tungsten, or other suitable flexible conductive foil materials. Further, in the illustrated non-limiting example, the dielectric film is a polymide film. In other aspects, the dielectric film could alternatively be made of high-dielectric constant but electrically insulating materials, such as LCP, polyester or some other insulating material that could be formed into a film. In an alternate embodiment, the flexible substrate could be made using conventional rigid board materials joined with flexible films.

Because of the flexible nature of the printed wiring 147 and the embedded capacitors 144, the local MRI coil 106 can be repeatedly folded and unfolded without damaging the mechanical properties (e.g., integrity, flexibility, elastic moduli, shape, etc.) or the electrical properties (e.g., radio-frequency, conductivity, permittivity, etc.) of the local MRI coil 106.

Referring now to FIG. 4, the MRI coil 106 is shown with an alternate tail section 152. The tail section 152 includes the tuning and matching capacitors 146, anti-parallel pin diode wafers 154, a micro-preamplifier wafer 156, and coaxial pads 158. The anti-parallel (“crossed”) pin diode wafers 154 are configured to protect the micro-preamplifier wafer 156 from damage when strong (≥0.5 Volt) rf pulses are received. The micro-preamplifier wafer 156 includes an rf-in pad 160, a chip pad 162, a ground pad 164, and an rf out pad 166. The micro-preamplifier 156 can amplify a received signal before the signal travels through the catheter 104, thereby effectively reducing electrical noise in the rf system 220 and increasing a signal to noise ratio of the local MRI coil 106. Each of the anti-parallel pin diode wafers 154 and the micro-preamplifier wafer 156 may further be covered to prevent water/liquid damage.

The preamplifier may be powered using direct-current (DC) power, which may be supplied by non-magnetic miniature rechargeable lithium-polymer batteries which are placed at the proximal end of the device (in the handle 102), and connected to the radio frequency line via a “Bias Tee” circuit, so that the DC power is then transported down the catheter shaft to the distal preamplifier over the same coaxial cable that is used to transport the radio frequency signal up the catheter shaft from the imaging coil.

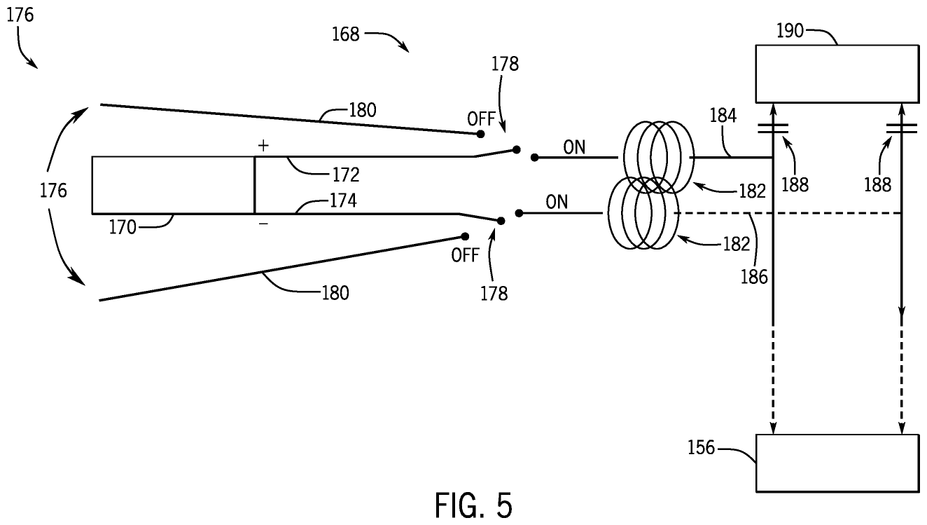

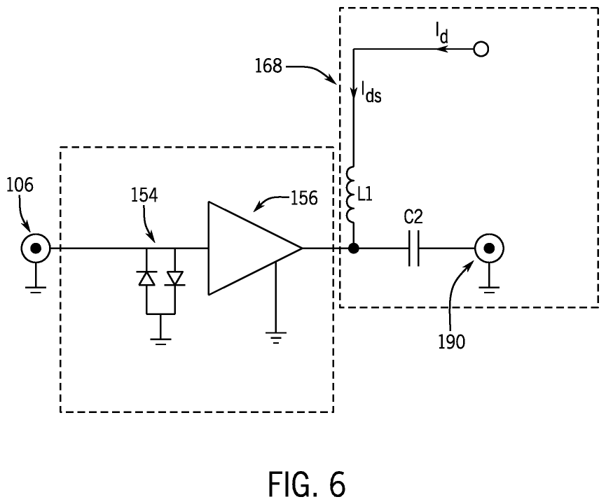

For example, as shown in FIG. 5, an MRI-compatible rechargeable DC powering circuit 168 can be constructed to power the preamplifier wafer 156. The DC powering circuit 168 is typically placed on the catheter handle 102, since it is too large to be placed inside the catheter shaft 120. The DC powering circuit 168 utilizes a rechargeable battery 170 to power the preamplifier wafer 156. In some instances, the battery 170 can be a rechargeable 3.7 Volt DC lithium-polymer battery. The battery 170 has two terminals, a positive (+) terminal 172 and a ground (-) terminal 174, which are connected to both the preamplifier wafer 156, as well as to a re-charging circuit 176 via a two-prong switch 178. Electrical leads 180 of the re-charging circuit 176 are typically connected to a USB port of a personal computer, which can supply the required charge to the battery 170, which can be, for example, 3.7 Volts. The mechanical switch 178 is used in order to switch the circuit 168 from an ON, preamplifier powering (working) mode, to an OFF, battery-charging (and non-powering) mode. Large inductors 182, which can be, for example, approximately 1 microHenry, are placed on both a conductor line 184 and a ground line 186 of the circuit 168 in order to prevent rf signals from entering the DC powering circuit 168. The circuit 168 is connected to the local MRI coil 106 using coaxial cables that run down the catheter shaft 120, and also serve to conduct the rf signal, acquired by the local MRI coil 106 up the catheter shaft, after the rf signal has been amplified by the preamplifier wafer 156. Additional components of the powering circuit are large blocking capacitors 188 (e.g., 1000 picoFarad), which serve to prevent DC voltage, coming from the powering circuit 168, from propagating towards a receiver of the rf system, which is opposed to the desired (powering) direction of the preamplifier wafer 156. FIG. 6 shows the typical circuit construct of the local MRI coil 106, the anti-parallel pin diode wafers 154, the preamplifier wafer 156, the DC powering circuit 168, and an MRI receiver 190.

Now that the various components of the expandable catheter device 100 have been discussed above, the general assembly of the components within the expandable catheter device 100 will be described below. It should be appreciated that the particular assembly configuration described is given as an example and is not limiting.

Referring now to FIG. 7A and 7B, the attachment configuration of the microfilaments 108 within the distal end 119 of the catheter 104 is illustrated. The microfilaments 108 are disposed between the outer sheath 122 and the inner shaft 120. A proximal end of each of the microfilaments 108 is attached to the outer sheath 122 near a proximal end of a corresponding one of the struts 128. A distal end of each of the microfilaments 108 is then attached to the outer sheath 122 near a distal end of a corresponding strut 128 that is arranged three struts 128 counter-clockwise of the strut 128 that the proximal end of the microfilament 108 is attached to. For example, if the five struts 128 are numbered as strut one through strut five traveling in a counter-clockwise direction around the circumference of the outer sheath 122 and the proximal end of the microfilament 108 is attached to the outer sheath 122 near a proximal end of strut one, then the distal end of the microfilament 108 is attached to the outer sheath 122 near a distal end of strut four. Correspondingly, the other four microfilaments 108 can be disposed between struts two and five, struts three and one, struts four and two, and struts five and three. This rotated attachment scheme aids in effectively collapsing the local MRI coil 106, as will be described below.

It should be appreciated that the number and rotated attachment scheme of the microfilaments 108 will correspond to at least one of the shape of the substrate 135 that the local MRI coil 106 is coupled to and the number of slits 126/struts 128 of the outer sheath 122. For example, a design with three slits 126/struts 128 may include three microfilaments 108, and the distal end of each of the microfilaments 108 may be attached near the distal end of a strut 128 that is arranged two struts 128 counter clockwise of a strut 128 that the proximal end of the microfilament 108 is attached to. A design with nine slits 126/struts 128 may include nine microfilaments 108, and the distal end of each of the microfilaments 108 may be attached near a distal end of a strut 128 that is arranged, for example, three to seven struts 128 counter clockwise of the strut 128 that the proximal end of the microfilament 108 is attached to. As the number of slits 126/struts 128 can continue toward larger numbers, it will be appreciated by those skilled in the art that any number of microfilaments 108 can be attached to the slits 126/struts 128 in any rotated attachment scheme that similarly aids in effectively collapsing the local MRI coil 106.

Additionally, in one aspect, the microfilaments 108 are attached to the outer sheath 122 by threading each microfilament 108 through a corresponding pre-drilled hole and locking the microfilament 108 in the pre-drilled hole using an adhesive. In other aspects, the microfilaments 108 can be attached to the outer sheath 122 using a variety of flexible adhesive materials, thermal welding, or ultrasonic welding, mechanical attachment such as with a rivet or fastener, or other suitable attachment methods.

Referring now to FIG. 8A, the distal end 119 of the catheter 104 is illustrated as fully assembled. The local MRI coil 106 and the tracking coils 110 are disposed within the distal end of the outer sheath 122. The tail section 136 of the substrate 135 is attached to one of the struts 128, such that a tip 150 (shown in FIG. 3) of the tail section 136 extends towards the proximal end 112 of the expandable catheter device 100. The strut attachment portions 138 are similarly attached to each of the four remaining struts 128. The microfilament attachment portions 140 of the substrate 135 are then attached to the microfilaments 108 described above. Additionally, the positional tracking (tracking) coils 110 are attached to the same struts 128 as the strut attachment portions 138, but are disposed more proximal than the strut attachment portions 138.

In the exemplary expandable catheter device 100, each of the tail section 136, the strut attachment portions 138, and the tracking coils 110 are attached to the struts 128 using an adhesive. In other examples, the various components can be attached to the struts 128 using a variety of elastically-flexible adhesive materials, thermal welding, ultrasonic welding, mechanical attachment such as with a rivet or fastener, or other suitable attachment methods. Additionally, in some other examples, the tracking coils 110 could be implemented into the local MRI coil 106 as additional flexible printed circuit also possessing embedded capacitors for purposes of tuning and matching. Each of the microfilament attachment portions 140 are attached to the microfilaments 108 by threading the microfilaments 108 through corresponding holes in the microfilament attachment portions 140. In some instances, the microfilaments 108 may then be secured within the microfilament attachment portions 140 using an adhesive to lock the microfilament attachment portion 140 relative to the corresponding microfilament 108. In other examples, the microfilament attachment portions 140 can be attached to the microfilaments 108 solely through the threaded connection, or through thermal welding, ultrasonic welding, mechanical attachment such as with a rivet or fastener, or other suitable attachment methods. The tracking coils 110 can have capacitors embedded therewith for tuning and matching, which provides great flexibility and precision compared to tuning and matching systems that are displaced from the coil, such as when located in the handle.

Having generally described the features of the expandable catheter device 100, a discussion of its general mode of operation is provided. By way of example, the operation of the expandable catheter device 100 will be described with respect to a cardiac atrial fibrillation procedure in which an RFA device is provided to the expandable catheter device 100 in order to provide thermal ablation therapy to a patient. As noted above, it should be appreciated by those skilled in the art that the expandable catheter device 100 can be employed for other procedures.

A target region of a left atrium is identified for treatment using an appropriate diagnostic procedure. Such procedures are well known in the art and are not described further herein. In the event that atrial ablation is desired, a physician makes a small incision in the body to gain access to a vascular pathway to the patient's heart. An initial guiding device, such as a guide wire, is used to guide the expandable catheter device 100 to the target region. This guide wire is separate from the expandable catheter device 100 and is used as a support for maneuvering the expandable catheter device 100 through the pathway to the target region. When the guide wire is in position, the expandable catheter device 100 is advanced so that the tip 150 of the expandable catheter device 100 is positioned proximate to the target region.

The physician manipulates the handle 102 of the expandable catheter device 100 in order to expand the catheter 104 to its expanded position. When the physician manipulates the handle 102, they move the sliding member 114 from the proximal position to the distal position. As the outer sheath 122 is rigidly attached to both the sliding member 114 at the proximal end and the inner shaft 120 at the distal end, when the sliding member 114 is moved to the distal position, the outer sheath 122 slides forward relative to the inner shaft 120, which then forces each of the five struts 128 at the distal end 119 of the expandable catheter device 100 to bend outwards, away from the inner shaft 120, as illustrated in FIGS. 1B, 7B, and 8A. As the struts 128 bend outwards, the microfilaments 108 become slack as the proximal and distal ends of the struts 128 are forced towards each other. Additionally, the tail section 136 and each of the strut attachment portions 138 of the substrate 135 are pulled apart by the struts 128, thereby expanding the local MRI coil 106.

Once the local MRI coil 106 is in its expanded position, it is operated to acquire image data, which, if the preamplifier wafer 156 is present, may include switching the switch 178 to the ON (working) position, so that the preamplifier is activated. From the acquired image data, images are reconstructed to confirm the location of the expandable catheter device 100 in relation to the target region. In this way, the physician has a visual means of tracking the precise location of the expandable catheter device 100. The tracking coils 110 may also acquire motion information and this information may be utilized to correct the acquired image data for motion effects. Once the expandable catheter device 100 is verified to be in the proper position using MRI, the guide wire is removed and an RFA device is advanced through the lumen 124 of the inner shaft 120. The RFA device is operated to deliver radio frequency (RF) energy to the target region to heat the target region in accordance with a treatment plan. During the ablation treatment, the local MRI coil 106 may acquire image data and images may be reconstructed. For example, images that depict the temperature of the target region can be reconstructed so that an accurate and real-time assessment of the efficacy of the ablation treatment can be assessed. Motion information may also be acquired at this time by the tracking coils 110 and this information may be utilized to correct the acquired image data for motion effects.

Methods for acquiring and reconstructing magnetic resonance images are well known in the art, including those methods for acquiring magnetic resonance images that depict temperature changes in tissue. Additionally, methods for acquiring and utilizing motion tracking information with magnetic resonance imaging are well known in the art. For example, magnetic resonance signals can be acquired and their phase information used to assess motion of the subject from which the signals originated. Exemplary methods for motion tracking and motion compensation include so-called “navigator-echo” methods. Generally, motion compensation may include both prospective and retrospective motion compensation. In prospective compensation techniques, the acquired motion tracking information is used to correct the acquired image data for motion artifacts prior to or during image reconstruction. In retrospective compensation techniques, the motion tracking information is used to selectively sort images after they have been reconstructed, for example, by sorting the images according to cardiac or respiratory phase. An added value of the local motion tracking coils 110 occurs in those situations in which various body tissues move at differing rates and when it is the goal of the targeted imaging to freeze the motion of the tissue-of-interest or region-of-interest alone. In such instances, the local motion tracking coils 110 are more sensitive to motion of the tissue of interest due to their proximity and physical contact with it, so they provide a better estimate of this motion that is possible with surface-based MRI techniques.

Upon completion of the ablation treatment, the RFA device is removed from the expandable catheter device 100 and optionally replaced with the guide wire. The physician then manipulates the handle 102 of the expandable catheter device 100 to collapse the catheter 104. When the physician manipulates the handle 102, the sliding member 114 is moved to the proximal position. This retracts the outer sheath 122, which in turn contracts the five struts 128 to the contracted position. When the outer sheath 122 is retracted, the microfilaments 108 are configured to slightly pull the microfilament attachment portions 140 of the substrate 135 towards the proximal end 112 of the expandable catheter device 100. This urges the substrate 135, as well as the local MRI coil 106, to bend at each of the five inner cutouts 142. The rotated attachment scheme of the microfilaments 108 pulls each of the five inner cutouts 142 in the same rotational direction, which aids in efficiently collapsing the local MRI coil 106 (as illustrated in FIGS. 8B and 8C). Once the expandable catheter device 100 is collapsed, it is then removed from the patient's heart and backed out through the pathway. If the guide wire was used again, it is then removed from the patient in a similar fashion.

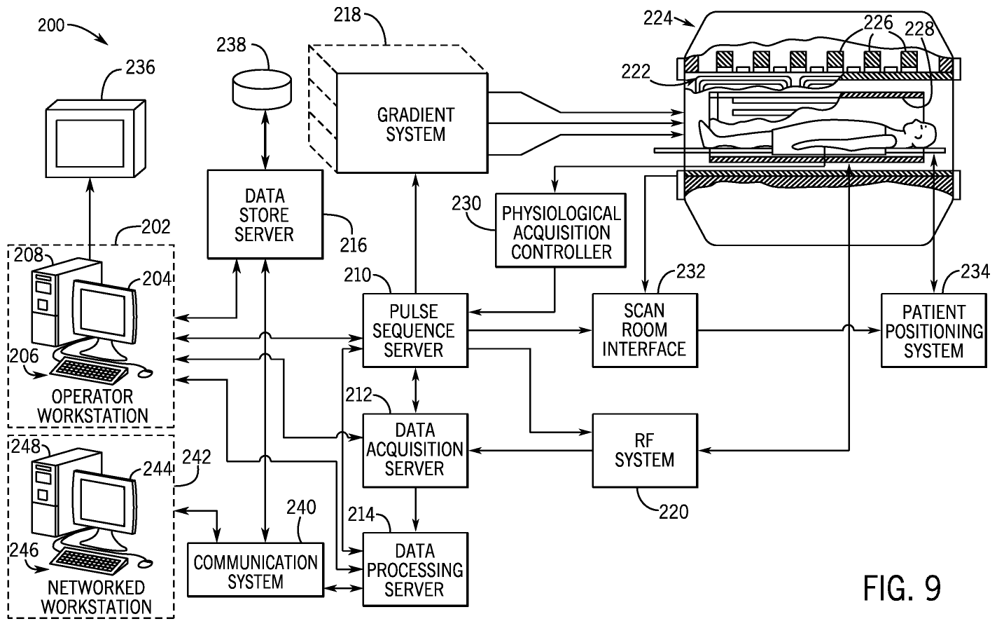

Referring to FIG. 9, an example of a MRI system 200 is provided that is configured for use with the above-described coil device and that can be used therewith to implement the methods described. The MRI system 200 includes an operator workstation 202 that may include a display 204, one or more input devices 206 (e.g., a keyboard, a mouse), and a processor 208. The processor 208 may include a commercially available programmable machine running a commercially available operating system. The operator workstation 202 provides an operator interface that facilitates entering scan parameters into the MRI system 200. The operator workstation 202 may be coupled to different servers, including, for example, a pulse sequence server 210, a data acquisition server 212, a data processing server 214, and a data store server 216. The operator workstation 202 and the servers 210, 212, 214, and 216 may be connected via a communication system 240, which may include wired or wireless network connections.

The pulse sequence server 210 functions in response to instructions provided by the operator workstation 202 to operate a gradient system 218 and a radiofrequency (“rf”) system 220. Gradient waveforms for performing a prescribed scan are produced and applied to the gradient system 218, which then excites gradient coils in an assembly 222 to produce the magnetic field gradients Gₓ, G_{y}, and G_{z} that are used for spatially encoding magnetic resonance signals. The gradient coil assembly 222 forms part of a magnet assembly 224 that includes a polarizing magnet 226, a whole-body RF coil 228, and the local MRI coil 106 (disposed within the illustrated patient).

rf waveforms are applied by the rf system 220 to the rf coil 228 or the local MRI coil 106, as will be the focus of the present disclosure, to perform the prescribed magnetic resonance pulse sequence. Responsive magnetic resonance signals detected by the rf coil 228, or the local MRI coil 106, are received by the rf system 220. The responsive magnetic resonance signals may be amplified, demodulated, filtered, and digitized under direction of commands produced by the pulse sequence server 210. The rf system 220 includes an rf transmitter for producing a wide variety of rf pulses used in MRI pulse sequences. The rf transmitter is responsive to the prescribed scan and direction from the pulse sequence server 210 to produce rf pulses of the desired frequency, phase, and pulse amplitude waveform. The generated rf pulses may be applied to the whole-body rf coil 228 or to one or more local coils, such as the local MRI coil 106, or coil arrays.

The rf system 220 also includes one or more rf receiver channels. An rf receiver channel includes an rf preamplifier that amplifies the magnetic resonance signal received by the coil (e.g., the whole-body rf coil 228 or the local MRI coil 106) to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

M=\sqrt{I²+Q²} (1);

and the phase of the received magnetic resonance signal may also be determined according to the following relationship:

$$ϕ = {tan}^{- 1} ( \frac{Q}{I} ) . \qquad ( 2 )$$

The pulse sequence server 210 may receive patient data from a physiological acquisition controller 230. By way of example, the physiological acquisition controller 230 may receive signals from a number of different sensors connected to the patient, including electrocardiograph (“ECG”) signals from electrodes, or respiratory signals from a respiratory bellows or other respiratory monitoring devices. These signals may be used by the pulse sequence server 210 to synchronize, or “gate,” the performance of the scan with the subject's heart beat or respiration.

The pulse sequence server 210 may also connect to a scan room interface circuit 232 that receives signals from various sensors associated with the condition of the patient and the magnet system. Through the scan room interface circuit 232, a patient positioning system 234 can receive commands to move the patient to desired positions during the scan.

The digitized magnetic resonance signal samples produced by the rf system 220 are received by the data acquisition server 212. The data acquisition server 212 operates in response to instructions downloaded from the operator workstation 202 to receive the real-time magnetic resonance data and provide buffer storage, so that data is not lost by data overrun. In some scans, the data acquisition server 212 passes the acquired magnetic resonance data to the data processor server 214. In scans that require information derived from acquired magnetic resonance data to control the further performance of the scan, the data acquisition server 212 may be programmed to produce such information and convey it to the pulse sequence server 210. For example, during pre-scans, magnetic resonance data may be acquired and used to calibrate the pulse sequence performed by the pulse sequence server 210. As another example, positional navigator (“navigator”) signals may be acquired and used to adjust the operating parameters of the rf system 220 or the gradient system 218, or to control the view order in which k-space is sampled. In still another example, the data acquisition server 212 may also process magnetic resonance signals used to detect the arrival of a contrast agent in a magnetic resonance angiography (“MRA”) scan. For example, the data acquisition server 212 may acquire magnetic resonance data and process it in real-time to produce information that is used to control the scan.

The data processing server 214 receives magnetic resonance data from the data acquisition server 212 and processes the magnetic resonance data in accordance with instructions provided by the operator workstation 202. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 214 are conveyed back to the operator workstation 202 for storage. Real-time images may be stored in a data base memory cache, from which they may be output to operator workstation 202 or a display 236. Batch mode images or selected real time images may be stored in a host database on disc storage 238. When such images have been reconstructed and transferred to storage, the data processing server 214 may notify the data store server 216 on the operator workstation 202. The operator workstation 202 may be used by an operator to archive the images, produce films, or send the images via a network to other facilities.

The MRI system 200 may also include one or more networked workstations 242. For example, a networked workstation 242 may include a display 244, one or more input devices 246 (e.g., a keyboard, a mouse), and a processor 248. The networked workstation 242 may be located within the same facility as the operator workstation 202, or in a different facility, such as a different healthcare institution or clinic.

The networked workstation 242 may gain remote access to the data processing server 214 or data store server 216 via the communication system 240. Accordingly, multiple networked workstations 242 may have access to the data processing server 214 and the data store server 216. In this manner, magnetic resonance data, reconstructed images, or other data may be exchanged between the data processing server 214 or the data store server 216 and the networked workstations 242, such that the data or images may be remotely processed by a networked workstation 242.

The present invention has been described in terms of one or more preferred embodiments, and it should be appreciated that many equivalents, alternatives, variations, and modifications, aside from those expressly stated, are possible and within the scope of the invention.

1. A catheter device for deploying a local magnetic resonance imaging coil, the catheter device comprising:
an inner shaft;
an outer sheath enveloping the inner shaft and including a plurality of slits extending in an axial direction proximate a distal end of the outer sheath, the slits separating a portion of the outer sheath into a plurality of struts;
a local magnetic resonance imaging coil disposed between the inner shaft and the outer sheath and coupled to the plurality of struts;
wherein moving the outer sheath relative to the inner shaft expands the catheter device from a contracted device position to an expanded device position; and
wherein when the catheter device is in the expanded position, the struts are bent radially outwards away from the inner shaft expanding the local magnetic resonance imaging coil from a contracted coil position to an expanded coil position.

2. The catheter device of claim 1 further comprising a handle including a sliding member rigidly fixed to the outer sheath.

3. The catheter device of claim 2 in which the sliding member is configured to move the outer sheath relative to the inner shaft to expand the catheter device from the contracted device position to the expanded device position.

4. The catheter device of claim 1 in which the inner shaft and the outer sheath are rigidly fixed relative to each other at a distal end of the catheter device.

5. The catheter device of claim 1 further comprising a plurality of microfilaments coupled to the outer sheath near a proximal end of the plurality of struts and near a distal end of the plurality of struts.

6. The catheter device of claim 5 in which the microfilaments are each coupled to the local magnetic resonance imaging coil and are configured to aid in collapsing the local magnetic resonance imaging coil when the local magnetic resonance imaging coil is moved from the expanded coil position to the contracted coil position.

7. The catheter device of claim 1 further comprising a plurality of motion tracking coils coupled to the plurality of struts.

8. The catheter device of claim 1 in which the local magnetic resonance imaging coil further includes tuning and matching capacitors.

9. The catheter device of claim 1 in which the local magnetic resonance imaging coil is configured to be in communication with a radio frequency (“rf”) system of a magnetic resonance imaging system.

10. The catheter device of claim 1 in which the local magnetic resonance imaging coil is coupled to a substrate, the substrate including a plurality of cutouts.

11. The catheter device of claim 10 in which each of the plurality of cutouts locally reduces a width of the substrate, providing a plurality of natural bend points in the substrate configured to aid in collapsing the local magnetic resonance imaging coil when the local magnetic resonance imaging coil is moved from the expanded coil position to the contracted coil position.

12. The catheter device of claim 1 in which the inner shaft further includes a lumen sized to receive a medical device.

13. The catheter device of claim 12 in which the medical device comprises at least one of a radio frequency ablation catheter, a laser ablation catheter, a cryoablation catheter, a stenting catheter, guide wires, and a balloon angioplasty catheter.

14. The catheter device of claim 1 in which the local magnetic resonance imaging coil further includes a preamplifier wafer and anti-parallel pin-diode wafers, the preamplifier wafer configured to reduce electrical noise in a radio frequency system and therefore substantially increase a signal to noise ratio of the local magnetic resonance imaging coil, and the anti-parallel pin-diode wafers configured to protect the preamplifier from being damaged during periods of strong radio-frequency signal reception.

15. A catheter device for deploying a local magnetic resonance imaging coil, the catheter device comprising:
an inner shaft;
an outer sheath including a plurality of slits extending in an axial direction proximate a distal end of the outer sheath, the slits separating a portion of the outer sheath into a plurality of struts;
a local magnetic resonance imaging coil disposed between the inner shaft and the outer sheath and coupled to the plurality of struts;
wherein moving the outer sheath relative to the inner shaft expands the catheter device from a contracted device position to an expanded device position.

16. The catheter device of claim 15 further comprising a handle including a sliding member rigidly fixed to the outer sheath.

17. The catheter device of claim 16 in which the sliding member is configured to move the outer sheath relative to the inner shaft to expand the catheter device from the contracted device position to the expanded device position.

18. The catheter device of claim 1 in which the inner shaft and the outer sheath are rigidly fixed relative to each other at a distal end of the catheter device.

19. The catheter device of claim 1 further comprising a plurality of microfilaments coupled to the outer sheath near a proximal end of the plurality of struts and near a distal end of the plurality of struts, each of the plurality of microfilaments being coupled to the local magnetic resonance imaging coil and configured to aid in collapsing the local magnetic resonance imaging coil when the local magnetic resonance imaging coil is moved from an expanded coil position to a contracted coil position.

20. The catheter device of claim 1 in which the local magnetic resonance imaging coil is coupled to a substrate, the substrate including a plurality of cutouts, which locally reduce a width of the substrate, providing a plurality of natural bend points in the substrate, which are configured to aid in collapsing the local magnetic resonance imaging coil when the local magnetic resonance imaging coil is moved from an expanded coil position to a contracted coil position.

21. The catheter device of claim 1 in which the inner shaft further includes a lumen sized to receive at least one of a radio frequency ablation catheter, a laser ablation catheter, a cryoablation catheter, a stenting catheter, a guide wire, and a balloon angioplasty catheter.
